(19) United States
(12) Patent Application Publication
(10) Pub. No.: US 2025/0099387 A1
(43) Pub. Date: Mar. 27, 2025

(54) POLYALKYLENE OXIDE PARTICLES FOR PHARMACEUTICAL FORMULATION, MEDICINAL COMPOSITION, COMPOSITION FOR PHARMACEUTICAL FORMULATION, AND PHARMACEUTICAL FORMULATION

(71) Applicant: SUMITOMO SEIKA CHEMICALS CO., LTD., Hyogo (JP)

(72) Inventors: Naoki SAINOU, Himeji-shi, Hyogo (JP); Shizuka HACHIKEN, Himeji-shi, Hyogo (JP)

(73) Assignee: SUMITOMO SEIKA CHEMICALS CO., LTD., Hyogo (JP)

(21) Appl. No.: 18/730,907

(22) PCT Filed: Jan. 27, 2023
(86) PCT No.: PCT/JP2023/002606

§ 371 Date: Jul. 22, 2024

(30) Foreign Application Priority Data
Jan. 31, 2022 (JP) 2022-013782

(51) Int. Cl. A61K 9/16

(52) U.S. Cl. CPC A61K 9/1641

(57) ABSTRACT

Provided are polyalkylene oxide particles suitable for use in forming preparations that have low friability and are less susceptible to chipping and cracking, a pharmaceutical composition comprising the polyalkylene oxide particles, and a preparation composition comprising the pharmaceutical composition.

In the polyalkylene oxide particles for a preparation of the present invention, the content of particles having a particle size of 150 μm or more is less than 46 mass %, and the content of particles having a particle size of 300 μm or more is less than 10 mass %. The pharmaceutical composition of the present invention comprises the polyalkylene oxide particles, and the preparation composition of the present invention comprises the pharmaceutical composition.

### TECHNICAL FIELD

The present invention relates to polyalkylene oxide particles for a preparation, a pharmaceutical composition, a preparation composition, and a preparation.

### BACKGROUND ART

Conventionally, polyalkylene oxide particles, typified by polyethylene oxide, are known to be used as binders for pharmaceutical preparations etc. For example, Patent Literature 1 discloses polyalkylene oxide particles with improved properties of mixing with various powder components contained in pharmaceutical preparations.

Polyalkylene oxide particles used for pharmaceutical preparations are excellent in compression molding properties and are thus widely used as binders for, for example, tablets.

### CITATION LIST

### Patent Literature

PTL 1: WO2012/165198

### SUMMARY OF INVENTION

### Technical Problem

However, in the production process of, for example, tablets, there are problems such as tablets being damaged due to friction etc. and tablets chipping and cracking due to collisions between tablets. There is thus a strong need to improve abrasion resistance (i.e., to suppress the increase in friability). In addition, it was found that reducing the coefficient of thermal expansion of compression-molded articles of polyalkylene oxide particles is also important in preventing tablets from chipping and cracking. If the coefficient of thermal expansion is high, cracking is likely to occur in the step of heating uncoated tablets. From these viewpoints, polyalkylene oxide particles as binders (excipients) are required to have properties to suppress friability and thermal expansion when preparations are formed in a dosage form such as tablets.

The present invention has been made in view of the above, and an object of the present invention is to provide polyalkylene oxide particles suitable for use in forming preparations that have low friability and are less susceptible to chipping and cracking, a pharmaceutical composition comprising the polyalkylene oxide particles, and a preparation composition comprising the pharmaceutical composition.

### Solution to Problem

The present inventors conducted extensive research to achieve the above object, and found that the above object can be achieved by polyalkylene oxide particles having a specific particle size distribution. Thus, the present invention has been accomplished.

Specifically, the present invention encompasses, for example, the subject matter of the following items.

### Item 1.

Polyalkylene oxide particles for a preparation,
wherein
the content of particles having a particle size of 150 μm or more is less than 46 mass %, and
the content of particles having a particle size of 300 μm or more is less than 10 mass %.

### Item 2.

The polyalkylene oxide particles for a preparation according to Item 1, which have a 1 mass % aqueous solution viscosity of 40 to 20000 mPa·s.

### Item 3.

The polyalkylene oxide particles for a preparation according to Item 1, which have a 1 mass % aqueous solution viscosity of less than 40 mPa·s and a 5 mass % aqueous solution viscosity of 30 to 50000 mPa·s.

### Item 4.

A pharmaceutical composition comprising the polyalkylene oxide particles for a preparation according to any one of Items 1 to 3.

### Item 5.

A preparation composition comprising the pharmaceutical composition according to Item 4.

### Item 6.

The preparation composition according to Item 5, which comprises the polyalkylene oxide particles in an amount of 20 mass or more.

### Item 7.

A preparation comprising the preparation composition according to Item 5 or 6.

### Item 8.

The preparation according to Item 7, which comprises a compression-molded article of the preparation composition.

### Advantageous Effects of Invention

The polyalkylene oxide particles of the present invention have a specific particle size distribution and are thus suitable for use in forming preparations that have low friability and are less susceptible to chipping and cracking. The pharmaceutical composition of the present invention comprises the polyalkylene oxide particles and is thus suitable for use as a raw material (preparation composition) for forming preparations that have low friability and are less susceptible to chipping and cracking.

### DESCRIPTION OF EMBODIMENTS

Embodiments of the present invention are described in detail below. In the present specification, the terms “comprising” and “containing” include the concepts of comprising, containing, consisting essentially of, and consisting of.

In the numerical range described in stages in the present specification, the upper or lower limit of the numerical range at one stage can be optionally combined with the upper or lower limit of the numerical range at another stage. In the numerical range described in the present specification, the upper or lower limit of the numerical range may be replaced with a value shown in the Examples or a value that can be uniquely derived from the Examples. Further, in the present specification, the numerical values connected by “to” mean the numerical range including the numerical values before and after “to” as the lower limit and the upper limit.

### 1. Polyalkylene Oxide Particles for Preparation

In the polyalkylene oxide particles for a preparation of the present invention (simply referred to below as “the polyalkylene oxide particles of the present invention” or “the polyalkylene oxide particles”), the content of particles having a particle size of 150 μm or more is less than 46 mass %, and the content of particles having a particle size of 300 μm or more is less than 10 mass %. That is, the polyalkylene oxide particles of the present invention are particles having a specific particle size distribution.

For example, when used as a binder (excipient) for forming preparations in a dosage form such as tablets, the polyalkylene oxide particles can impart low friability to the preparations, making them less susceptible to chipping and cracking. Moreover, a compression-molded article of the polyalkylene oxide particles of the present invention has a low coefficient of thermal expansion, which makes it possible to provide preparations that are less susceptible to chipping and cracking. Thus, the polyalkylene oxide particles of the present invention are suitable for use in forming preparations that have low friability and are less susceptible to chipping and cracking.

The polyalkylene oxide particles have a particle form, and the type is not particularly limited as long as the polyalkylene oxide particles have the particle size distribution described above.

In the polyalkylene oxide particles, the type of polyalkylene oxide is not particularly limited, and examples include a wide range of known polyalkylene oxides.

In the polyalkylene oxide, the number of carbon atoms in the alkylene moiety is preferably 2 or more and 4 or less, for example. Because the effects of the present invention can be easily exhibited, it is particularly preferable that the alkylene moiety has 2 carbon atoms; that is, the polyalkylene oxide is polyethylene oxide. Specific examples of polyalkylene oxides other than polyethylene oxide include polypropylene oxide, polybutylene oxide, ethylene oxide/propylene oxide copolymers, ethylene oxide/butylene oxide copolymers, and the like.

The polyalkylene oxide is generally a homopolymer, but is not limited to this, and may be a copolymer. When the polyalkylene oxide is a copolymer, the polyalkylene oxide has, for example, two or more structural units with alkylene moieties having different numbers of carbon atoms.

The alkylene moiety of the polyalkylene oxide particles preferably contains at least an ethylene oxide unit, in terms of ease of production and imparting lower friability to preparations. That is, in the polyalkylene oxide particles, the polyalkylene oxide is preferably polyethylene oxide, or a copolymer of an ethylene oxide unit and other units (e.g., an ethylene oxide/propylene oxide copolymer or an ethylene oxide/butylene oxide copolymer, mentioned above).

The polyalkylene oxide particles may contain one type of polyalkylene oxide, or two or more types of polyalkylene oxides.

As described above, in the polyalkylene oxide particles, the content of particles having a particle size of 150 μm or more is less than 46 mass %, and the content of particles having a particle size of 300 μm or more is less than 10 mass %. This allows the polyalkylene oxide particles to impart lower friability to preparations and also allows a compression-molded article of the polyalkylene oxide particles to have a low coefficient of thermal expansion.

In the polyalkylene oxide particles, the content of particles having a particle size of 150 μm or more is preferably 43 mass % or less, more preferably 40 mass % or less, even more preferably 35 mass % or less, and particularly preferably 30 mass or less. The lower limit of the content of particles having a particle size of 150 μm or more in the polyalkylene oxide particles is not particularly limited, and is preferably 5 mass % or more in terms of high flowability.

In the polyalkylene oxide particles, the content of particles having a particle size of 300 μm or more is preferably 9 mass % or less, more preferably 7 mass % or less, even more preferably 5 mass % or less, and particularly preferably 3 mass % or less. The lower limit of the content of particles having a particle size of 300 μm or more in the polyalkylene oxide particles is not particularly limited, and may be, for example, 0 mass %.

The content of polyalkylene oxide particles having a particle size of 150 μm or more in the polyalkylene oxide particles can be calculated by classifying the polyalkylene oxide particles using a sieve with a mesh opening of 150 μm (JIS Z 8801-1 standard sieve). Specifically, the polyalkylene oxide particles are classified using a sieve with a mesh opening of 150 μm, the mass of polyalkylene oxide particles remaining on the sieve is measured, and the proportion thereof based on the total mass of the polyalkylene oxide particles used in classification is calculated to thereby determine the content of polyalkylene oxide particles having a particle size of 150 μm or more. As is clear from this explanation, the phrase “polyalkylene oxide particles having a particle size of 150 μm or more” refers to particles remaining on the sieve after the polyalkylene oxide particles are classified using a sieve with a mesh opening of 150 μm.

The content of polyalkylene oxide particles having a particle size of 300 μm or more in the polyalkylene oxide particles can be calculated by classifying the polyalkylene oxide particles using a sieve with a mesh opening of 300 μm (JIS Z 8801-1 standard sieve). Specifically, the polyalkylene oxide particles are classified using a sieve with a mesh opening of 300 μm, the mass of polyalkylene oxide particles remaining on the sieve is measured, and the proportion thereof based on the total mass of the polyalkylene oxide particles used in classification is calculated to thereby determine the content of polyalkylene oxide particles having a particle size of 300 μm or more. As is clear from this explanation, the phrase “polyalkylene oxide particles having a particle size of 300 μm or more” refers to particles remaining on the sieve after the polyalkylene oxide particles are classified using a sieve with a mesh opening of 300 μm.

In the polyalkylene oxide particles, the content of polyalkylene oxide particles having a particle size of 500 μm or more is preferably 5 mass % or less, more preferably 1 mass % or less, and even more preferably 0 mass % (i.e., not containing polyalkylene oxide particles having a particle size of 500 μm or more).

The method for adjusting the particle size distribution (e.g., the proportions of particles having particle sizes of 150 μm or more and 300 μm or more) of the polyalkylene oxide particles is not particularly limited. For example, known particle size adjustment methods can be widely used. Examples of the adjustment method include a method for adjusting the particle size distribution in the process of producing polyalkylene oxide particles, and a method for adjusting the particle size distribution by classification or the like of the resulting polyalkylene oxide particles.

In an embodiment of the method for adjusting the particle size distribution, polyalkylene oxide particles obtained by a known production method or commercially available polyalkylene oxide particles are classified using a sieve with a mesh opening of 150 μm, and particles remaining on the sieve and particles passing though the sieve are mixed at a specific ratio, thereby adjusting the content of polyalkylene oxide particles having a particle size of 150 μm or more. The adjustment method according to the present embodiment can easily adjust the content of polyalkylene oxide particles having a particle size of 150 μm or more to a desired range. The content of polyalkylene oxide particles having a particle size of 300 μm or more can also be adjusted in a similar manner.

Other examples of the method for adjusting the particle size distribution include methods of adjusting the type of raw materials (an alkylene oxide, a catalyst, etc.) used when producing polyalkylene oxide particles, the proportions of raw materials, polymerization temperature, polymerization time, the amount of solvent used, and other conditions.

In terms of easily imparting low friability to preparations, the polyalkylene oxide particles preferably have a 1 mass % aqueous solution viscosity of 40 to 20000 mPa·s. The 1 mass % aqueous solution viscosity is more preferably 1000 mPa·s or more, and is also more preferably 13000 mPa·s or less, and even more preferably 10000 mPa·s or less.

The polyalkylene oxide particles may have a 1 mass % aqueous solution viscosity of less than 40 mPa·s; however, in this case, the polyalkylene oxide particles have a 5 mass % aqueous solution viscosity of 30 to 50000 mPa·s. That is, in an embodiment of the polyalkylene oxide particles of the present invention, the polyalkylene oxide particles may have a 1 mass % aqueous solution viscosity of less than 40 mPa·s and a 5 mass % aqueous solution viscosity of 30 to 50000 mPa·s. Also due to this, the polyalkylene oxide particles can impart lower friability to preparations. The 5 mass % aqueous solution viscosity of the polyalkylene oxide particles is more preferably 40 mPa·s or more, and even more preferably 50 mPa·s or more. The 1 mass % aqueous solution viscosity and 5 mass % aqueous solution viscosity of the polyalkylene oxide particles can be measured using a rotational viscometer (RV DVII+, produced by Brookfield).

The aerated bulk density of the polyalkylene oxide particles is preferably 0.15 to 0.60 g/mL. In this case, production efficiency and transport efficiency are excellent, and the formability of preparations tends to be good. The aerated bulk density of the polyalkylene oxide particles is more preferably 0.20 to 0.55 g/mL. In the present invention, the aerated bulk density of the polyalkylene oxide particles refers to a value measured according to JIS K6720 4.3.

The mass average molecular weight of the polyalkylene oxide particles is not particularly limited. In terms of easily imparting low friability to preparations and reducing the coefficient of thermal expansion, the mass average molecular weight of the polyalkylene oxide particles is preferably 100000 or more and 15 million or less. The mass average molecular weight of the polyalkylene oxide particles is more preferably 200000 to 12 million, even more preferably 2 million to 10 million, and still even more preferably 3 million to 8 million. The mass average molecular weight of the polyalkylene oxide as mentioned herein refers to a value measured by gel permeation chromatography, and particularly refers to a value calculated from a calibration curve created using a known polyethylene oxide standard sample.

The form of the polyalkylene oxide particles is not particularly limited, and may be, for example, spherical, ellipsoidal, or amorphous.

The degree of dispersion of the polyalkylene oxide particles is not particularly limited, and is preferably, for example, 10 to 20%. Thus, the polyalkylene oxide particles have suitable dispersibility, which makes it easy to handle the powder, and reproducibility during the production of preparations is good. This makes it easy to provide a preparation that has low friability and also makes it easy to provide a preparation in which a compression-molded article has a lower coefficient of thermal expansion.

The degree of compression of the polyalkylene oxide particles is not particularly limited, and is preferably, for example, 14 to 24%. Thus, the polyalkylene oxide particles have suitable compressibility, which makes it easy to provide a preparation that has low friability and also makes it easy to provide a preparation in which a compression-molded article has a lower coefficient of thermal expansion.

The polyalkylene oxide particles have the particle size distribution described above. In short, since the proportion of particles having a coarse particle size is controlled to be below a certain level, the polyalkylene oxide particles have excellent compression molding properties, and can impart low friability to preparations especially when used as an excipient for forming preparations in a dosage form such as tablets.

In addition, in the polyalkylene oxide particles of the present invention, the coefficient of thermal expansion of a compression-molded article is low. Thus, when the polyalkylene oxide particles are used as an excipient for producing preparations, cracking or the like is less likely to occur in the process of heating uncoated tablets, and as a result, preparations in which chipping and cracking are suppressed can be obtained.

When used as an excipient for preparations, the polyalkylene oxide particles of the present invention can prevent abrasion of preparations during the production process without changing the composition of the preparations, and preparations having excellent abrasion resistance can be industrially produced stably.

Thus, the polyalkylene oxide particles of the present invention can be suitably used as a raw material, in particular, an excipient, for producing preparations that have low friability and are less susceptible to chipping and cracking. That is, the polyalkylene oxide particles of the present invention are preferable for pharmaceuticals and preparations, and particularly preferable as an excipient for tablets.

### 2. Method for Producing Polyalkylene Oxide Particles

As the method for producing the polyalkylene oxide particles of the present invention, for example, methods for producing known polyalkylene oxide particles can be widely used.

For example, the polyalkylene oxide particles can be obtained by polymerization reaction of an alkylene oxide in the presence of an alkali or a metal catalyst. Examples of the alkylene oxide used herein include aliphatic alkylene oxides. Specific examples include ethylene oxide, propylene oxide, and butylene oxide; preferably ethylene oxide or propylene oxide; and particularly preferably ethylene oxide. Alkylene oxides can be used singly or in combination of two or more.

The catalyst can be, for example, an alkali catalyst or a metal catalyst. As the metal catalyst, for example, metal catalysts conventionally used in the production of polyalkylene oxide can be widely used. Of these, an organic zinc catalyst is preferred. Organic zinc catalysts can be obtained by known production methods, preferably by step C of reacting an organic zinc compound with an aliphatic polyhydric alcohol and a monohydric alcohol in a solvent to form a particulate reaction product (organic zinc catalyst).

When an organic zinc compound is reacted with a monohydric alcohol and an aliphatic polyhydric alcohol in step C, it is preferable to use a reaction solvent to allow the reaction to smoothly proceed. In this case, the amount of solvent used is preferably 200 to 1500 parts by mass, more preferably 300 to 1200 parts by mass, and even more preferably 400 to 1000 parts by mass, per 100 parts by mass of the organic zinc compound, from the viewpoint of economy and suppression of reaction heat. The solvent is not particularly limited. Examples include aliphatic hydrocarbons, such as 2-methylpentane, n-pentane, n-hexane, n-heptane, isopentane, and cyclohexane; aromatic hydrocarbons, such as benzene, toluene, and xylene; and the like. From the viewpoint of efficient production of an alkylene oxide polymer, it is preferred that the solvent used for the preparation of the zinc catalyst in step C and the inert hydrocarbon solvent used for the polymerization reaction of the alkylene oxide are the same.

The aliphatic polyhydric alcohol used in step C is an aliphatic polyhydric alcohol having two or more carbon atoms and two or more hydroxyl groups in the molecule. Specific examples of aliphatic polyhydric alcohols include ethylene glycol, propylene glycol, 1,2-butanediol, 1,3-butanediol, 1,4-butanediol, 2,3-butanediol, 1,5-pentanediol, 2,3,4-pentanetriol, 1,6-hexanediol, glycerin, pentaerythritol, and the like. Among these, from the viewpoint of the activity of the resulting zinc catalyst, aliphatic polyhydric alcohols having four carbon atoms are preferable, and specifically, 1,3-butanediol and 1,4-butanediol can be suitably used.

In step C, the reaction is preferably carried out under stirring conditions at a tip peripheral speed of 1.5 m/sec or more. The catalyst obtained in this case facilitates production of polyalkylene oxide particles with which preparations that have low friability can be easily formed. In the reaction of step C, a method of reacting a portion of the monohydric alcohol and then reacting the monohydric alcohol and the aliphatic alcohol simultaneously is preferred. The catalyst obtained in this case also facilitates production of polyalkylene oxide particles with which preparations that have low friability can be easily formed.

The zinc content of the organic zinc catalyst is preferably 0.5 to 10 mass %, and more preferably 1.0 to 5.0 mass %, in terms of, for example, facilitating production of polyalkylene oxide particles with which preparations that have low friability can be easily formed.

The amount of catalyst used may be the same as that in methods for producing known polyalkylene oxide particles. For example, the amount of catalyst used may be a catalytic amount.

The polymerization reaction of the alkylene oxide can be performed in a solvent. As such solvents, those used in methods for producing known polyalkylene oxides can be widely used. Examples include at least one hydrocarbon solvent selected from the group consisting of 2-methylpentane, n-pentane, n-hexane, n-heptane, isopentane, and cyclohexane. N-hexane or n-pentane is preferably used because they are easily available industrially, and because they have a boiling point lower than the melting point of the resulting polyalkylene oxide and are easy to remove after the polymerization reaction. The amount of polymerization solvent used is preferably 100 to 10000 parts by mass, more preferably 200 to 2000 parts by mass, even more preferably 410 to 1000 parts by mass, still even more preferably 420 to 800 parts by mass, and particularly preferably 450 to 600 parts by mass, per 100 parts by mass of alkylene oxide, in terms of removing the heat of polymerization and easily controlling the polymerization reaction.

In the polymerization reaction of the alkylene oxide, the ratio of the alkylene oxide to the solvent (alkylene oxide/solvent) is preferably 0.23 or less. In the polymerization reaction of the alkylene oxide, the ratio of the solvent added relative to the volume of the reaction vessel is preferably 30% or less. In either case, it is easy to produce polyalkylene oxide particles with which preparations that have low friability can be easily formed.

A chain transfer agent can be used in the polymerization reaction of the alkylene oxide, as in, for example, methods for producing known polyalkylene oxide particles.

### 3. Pharmaceutical Composition

The pharmaceutical composition of the present invention comprises the polyalkylene oxide particles for a preparation of the present invention described above. Thus, when used as an excipient for forming preparations in a dosage form such as tablets, the pharmaceutical composition of the present invention can impart low friability to the preparations, and can be suitably used as a raw material, in particular, an excipient, for producing preparations that are less susceptible to chipping and cracking. That is, the pharmaceutical composition of the present invention is suitable as a raw material for preparing preparation compositions.

The pharmaceutical composition of the present invention may consist of the polyalkylene oxide particles or may contain components other than the polyalkylene oxide particles.

### 4. Preparation Composition

The preparation composition of the present invention comprises the pharmaceutical composition of the present invention described above. Specifically, the pharmaceutical composition of the present invention may comprise the polyalkylene oxide particles and components other than the polyalkylene oxide particles.

As components other than the polyalkylene oxide particles (referred to below as “other components”), for example, various components contained in known preparation compositions can be widely applied. Specific examples of other components include active components, fillers, excipients other than polyalkylene oxide particles, diluents, lubricants, dyes, pigments, coloring agents, osmotic agents, and the like.

The preparation composition of the present invention may contain the polyalkylene oxide particles and silica as a filler. When the preparation composition of the present invention contains the polyalkylene oxide particles and silica, the preparation composition of the present invention preferably contains silica in an amount of 3.0 parts by mass or less per 100 parts by mass of the polyalkylene oxide particles. This tends to enhance the flowability etc. of the polyalkylene oxide particles. The content of silica per 100 parts by mass of the polyalkylene oxide particles is, for example, more preferably 2.0 parts by mass or less, and even more preferably 1.5 parts by mass or less. In terms of easily enhancing the flowability etc., the content of silica per 100 parts by mass of the polyalkylene oxide particles is, for example, more preferably 0.1 parts by mass or more.

As silica, for example, known silica can be widely used. Specifically, Aerosil and the like can be used.

The content of the polyalkylene oxide particles in the preparation composition is not particularly limited as long as the effects of the present invention are not impaired. In terms of easily imparting particularly low friability to preparations, the preparation composition preferably contains the polyalkylene oxide particles in an amount of 20 mass % or more, preferably 30 mass % or more, more preferably 40 mass % or more, and even more preferably 50 mass % or more, based on the total mass of the polyalkylene oxide particles and the other components (or the total mass of the preparation composition). Further, the polyalkylene oxide particles are preferably contained in an amount of 90 mass % or less based on the total mass of the polyalkylene oxide particles and the other components (or the total mass of the preparation composition).

In particular, the preparation composition of the present invention contains the polyalkylene oxide particles, and thus has excellent compression molding properties, and a compression-molded article can be easily obtained. The method for obtaining the compression-molded article is not particularly limited. For example, known compression molding methods can be widely used.

The method for preparing the preparation composition of the present invention is not particularly limited, and can be the same as, for example, methods for preparing known preparation compositions. For example, the preparation composition can be prepared by mixing polyalkylene oxide particles and a filler (e.g., silica) to obtain, for example, filler-coated polyalkylene oxide particles, and then mixing the polyalkylene oxide particles with various other components at a specific ratio.

The preparation composition of the present invention can be used to prepare various preparations. Since such preparations contain the preparation composition of the present invention, i.e., the polyalkylene oxide particles, the preparations have low friability.

The preparation of the present invention may contain a compression-molded article of the preparation composition. In this case, the preparation can be formed into various dosage forms, including compression-molded articles. Examples include tablets.

The features (properties, structures, functions, etc.) described in the embodiments of the present disclosure may be combined in any manner to specify the subject matter included in the present disclosure. That is, this disclosure includes all of the subject matter comprising any combination of the combinable features described herein.

### EXAMPLES

The present invention is described in more detail below with reference to Examples; however, the present invention is not limited to these Examples.

### Production Example 1

A 500 mL round-bottom flask with an inner diameter of 80 mm equipped with a cooling device, a dropping funnel, a nitrogen gas inlet tube, and an impeller with four paddle blades with a blade diameter of 53 mm (inclined at 45 degrees) as a stirrer was prepared. After the inside of the flask was purged with nitrogen, 87.2 g of n-hexane (produced by Sumitomo Chemical Company, Limited; high purity for industrial use) and 9.90 g of diethyl zinc (produced by Nippon Aluminum Alkyls, Ltd.) were placed in the flask. With stirring at a tip peripheral speed of 1.94 m/sec (stirring speed: 700 rpm) at an internal temperature of 20° C., as the first stage, reaction was performed while adding 1.47 g (0.032 mol) of ethyl alcohol (EtOH) dropwise at 0.2 g/min. As the second stage, a mixed liquid of 6.49 g (0.072 mol) of 1,4-butanediol (1,4-BDO) and 13.27 g (0.288 mol) of ethyl alcohol was added dropwise at 0.2 g/min to the reaction liquid cooled to an internal temperature of 10° C. After completion of the dropwise addition, the inside of the flask was heated to 30° C., and reaction was performed for 1 hour. Then, the temperature was raised to 50° C., and reaction was performed for 1 hour. Thereafter, heating was performed at an oil bath temperature of 80° C., and unreacted components were removed by distillation. After distillation, the inside of the flask was allowed to cool to room temperature, and 52.4 g of n-hexane was added, followed by heating at an oil bath temperature of 80° C. to perform the second distillation. This operation was repeated one more time, and a total of three distillations were performed. After the third distillation and then cooling, the resultant was diluted with 262 g of n-hexane and transferred to a pressure-resistant vessel sufficiently purged with nitrogen to obtain a zinc catalyst A (295 g) containing 1.8 mass % zinc.

### Production Example 2

A 500 mL round-bottom flask with an inner diameter of 80 mm equipped with a cooling device, a dropping funnel, a nitrogen gas inlet tube, and an impeller with four paddle blades with a blade diameter of 53 mm (inclined at 45 degrees) as a stirrer was prepared. The flask was purged with nitrogen, and 87.2 g of n-hexane and 9.90 g of diethyl zinc (produced by Nippon Aluminum Alkyls, Ltd.) were placed in the flask. With stirring at a tip peripheral speed of 0.97 m/sec (stirring speed: 350 rpm) at an internal temperature of 20° C., as the first stage, reaction was performed while adding 11.03 g (0.239 mol) of ethyl alcohol (EtOH) dropwise at 0.2 g/min. As the second stage, a mixed liquid of 6.49 g (0.072 mol) of 1,4-butanediol (1,4-BDO) and 13.27 g (0.288 mol) of ethyl alcohol was added dropwise at 0.2 g/min to the reaction liquid cooled to an internal temperature of 10° C. After completion of the dropwise addition, the inside of the flask was heated to 30° C., and reaction was performed for 1 hour. Then, the temperature was raised to 50° C., and reaction was performed for 1 hour. Thereafter, heat treatment was performed at an oil bath temperature of 130° C. for 15 minutes without sealing. After cooling, the resultant was diluted with 262 g of n-hexane and transferred to a pressure-resistant vessel sufficiently purged with nitrogen to obtain a zinc catalyst B (270 g) containing 1.9 mass % zinc.

### Example 1

The inside of a 1 L pressure-resistant reaction vessel with an inner diameter of 94 mm equipped with a dropping funnel, a nitrogen gas inlet tube, and an anchor impeller with a blade diameter of 47 mm as a stirrer was sufficiently purged with nitrogen. Subsequently, 271 g of n-hexane was placed in the vessel, and 1.405 g of the zinc catalyst A (0.0004 mol in terms of zinc) obtained in Production Example 1 was uniformly dispersed. Then, 58.0 g (1.32 mol) of ethylene oxide was added, the vessel was sealed, and polymerization was performed with stirring in a thermostatic bath at 40° C. After completion of the polymerization, the white product was filtered out, dried under reduced pressure at 40° C., and then passed through a JIS Z 8801-1 standard sieve (500 μm) to obtain 43.3 g of polyethylene oxide particles. The viscosity of a 1 mass % aqueous solution of the obtained polyethylene oxide particles was 11900 mPa·s.

### Example 2

41.3 g of polyethylene oxide particles were obtained in the same manner as in Example 1, except that the amount of the zinc catalyst A used was 2.810 g (0.0008 mol in terms of zinc), and the amount of ethylene oxide used was 50.0 g (1.14 mol). The viscosity of a 1 mass % aqueous solution of the obtained polyethylene oxide particles was 8000 mPa·s.

### Comparative Example 1

59 g of polyethylene oxide particles were obtained in the same manner as in Example 1, except that the amount of the zinc catalyst A used was 2.108 g (0.0006 mol in terms of zinc), the amount of n-hexane was 345 g, and the amount of ethylene oxide used was 85 g (1.93 mol). The viscosity of a 1 mass % aqueous solution of the obtained polyethylene oxide particles was 10980 mPa·s.

### Comparative Example 2

59.0 g of polyethylene oxide particles were obtained in the same manner as in Comparative Example 1, except that 1.262 g (0.0004 mol in terms of zinc) of the zinc catalyst B was used in place of the zinc catalyst A, and the amount of n-hexane was 271 g. The viscosity of a 1 mass % aqueous solution of the obtained polyethylene oxide particles was 15560 mPa·s.

### Comparative Example 3

18.0 g of polyethylene oxide particles were obtained in the same manner as in Example 1, except that the amount of the zinc catalyst A used was 2.810 g (0.0008 mol in terms of zinc), the amount of ethylene oxide used was 82.8 g (1.88 mol), and the mesh opening of the JIS Z 8801-1 standard sieve was 1000 μm. The viscosity of a 1 mass % aqueous solution of the obtained polyethylene oxide particles was 16060 mPa·s.

### Comparative Example 4

50.3 g of polyethylene oxide particles were obtained in the same manner as in Example 1, except that the amount of the zinc catalyst A used was 2.108 g (0.0006 mol in terms of zinc), the amount of ethylene oxide used was 79.2 g (1.80 mol), and the mesh opening of the JIS Z 8801-1 standard sieve was 1000 μm. The obtained polyethylene oxide particles were classified using JIS Z 8801-1 standard sieves (a sieve with a mesh opening of 500 μm, a sieve with a mesh opening of 300 μm, a sieve with a mesh opening of 250 μm, a sieve with a mesh opening of 180 μm, a sieve with a mesh opening of 150 μm, a sieve with a mesh opening of 106 μm, a sieve with a mesh opening of 75 μm, and a saucer), and adjustment was performed so that the particle size distribution was as shown in Table 1 shown below. The viscosity of a 1 mass % aqueous solution of the obtained polyethylene oxide particles was 16040 mPa·s.

### Evaluation Methods

### 1 Mass % Aqueous Solution Viscosity of Polyalkylene Oxide

6 g of polyethylene oxide particles and 125 mL of isopropanol were added to a 1 L beaker, and while stirring at 350 rpm using an impeller, 594 g of ion-exchange water was added, and the mixture was stirred for 1 minute. Then, the stirring speed was changed to 60 rpm, and stirring was further continued for 3 hours, thereby obtaining a 1 mass % aqueous solution of polyethylene oxide. The aqueous solution was maintained at 25° C., the viscosity was measured using a rotational viscometer (RV DVII+, produced by Brookfield; spindle: RV-2, rotation speed: 2 rpm), and this value was taken as the 1 mass % aqueous solution viscosity.

### Method for Measuring Particle Size Distribution (Mass Percentage of Particles on Each Sieve)

The particle size distribution of polyethylene oxide particles was measured and calculated by a dry sieving test. First, polyethylene oxide particles were mixed with 1 mass % amorphous silica (Aerosil produced by Nippon Aerosil Co., Ltd.) as a lubricant to prepare a mixture. As JIS Z 8801-1 standard sieves, a sieve with a mesh opening of 500 μm, a sieve with a mesh opening of 300 μm, a sieve with a mesh opening of 250 μm, a sieve with a mesh opening of 180 μm, a sieve with a mesh opening of 150 μm, a sieve with a mesh opening of 106 μm, and a sieve with a mesh opening of 75 μm were stacked on a saucer in this order from above. The mixture was put in the uppermost sieve with a mesh opening of 500 μm, and the sieves were shaken using a rotating-tapping shaker for 20 minutes to classify the mixture. After classification, the mass of polyalkylene oxide particles remaining on each sieve was measured, and the percentage of each mass relative to the total mass was calculated and used as the particle size distribution of the polyalkylene oxide particles.

### Measurement of Friability

A mixture obtained by mixing 11.736 g of the polyethylene oxide particles obtained in each of the Examples and the Comparative Examples with 0.12 g of amorphous silica (Aerosil produced by Nippon Aerosil Co., Ltd.), 0.168 g of iron sesquioxide (Fe₂O₃), 4.98 g of sodium chloride having a particle size of less than 150 μm as an osmotic agent, and 0.036 g of magnesium stearate as a lubricant were uniformly mixed in a mortar with a pestle to obtain a preparation composition. 426 mg of the preparation composition was put into a general-purpose autograph mortar (produced by Ichihashi Seiki Co., Ltd., Φ10, R10), and compression-molded using an Autograph (AGS-T, produced by Shimadzu Corporation) with a test force of 5 kN at a compression speed of 100 mm/min to obtain a tablet. The same compression molding operation was repeated to produce a total of 8 tablets for each preparation composition.

The friability of the tablets obtained was determined using a Friabimat SA-400 (produced by Copley Scientific). Each tablet was vacuum-dried and then sealed in a special glass container. The glass container was attached to the vibration portion of the device, and a test was performed under conditions of a vibration frequency of 400 times/min and a test time of 2400 seconds. The friability was calculated from the tablet mass before and after the test using the following formula (1).

$$Friability [ \% ] = \{ ( M 1 - M 2 ) / M 1 \} \times 100 \qquad ( 1 )$$

(In formula (1), M1 is the tablet mass before the test, and M2 is the tablet mass after the test.)

M1 and M2 were weighed on an electronic balance (HR-200 produced by A&D Company, Limited), and before weighing, the tablets were blown with nitrogen for about 5 to 10 minutes to remove powder adhering to the tablet surface and to dry the tablets.

### Degree of Dispersion

The degree of dispersion of polyalkylene oxide particles was measured with a Powder Tester PT-X (produced by Hosokawa Micron Corporation).

### Degree of Compression

A 20 mL graduated glass cylinder was filled with 10 mL or more of polyalkylene oxide particles and freely dropped from a height of 4 cm for tapping. The aerated bulk density and the packed bulk density were calculated from the weights and volumes of the polyalkylene oxide particles after 0 and 200 tappings, and the degree of compression was determined.

### Coefficient of Thermal Expansion

426 mg of polyalkylene oxide particles were put into a general-purpose autograph mortar ($11, flat) and compressed using an Autograph (AGS-T, produced by Shimadzu Corporation) with a test force of 5 kN at a compression speed of 10 mm/min to obtain a tablet of polyethylene oxide particles alone (compression-molded article). This was allowed to stand at room temperature for a sufficient time, and the thickness D1 of the compression-molded article was measured. Thereafter, the compression-molded article was allowed to stand in a blow dryer (FV-320 produced by Advantec) set at 40° C. for 2 hours, and the thickness D2 of the compression-molded article was measured. Then, the coefficient of thermal expansion (%) of the compression-molded article was determined using the following formula (2).

$$Coefficient of Thermal Expansion = \{ ( D 2 - D 1 ) / ( D 1 ) \} \times 100 \qquad ( 2 )$$

TABLE 1

| | | | | | | | | | Proportion | Proportion | | | | | |
|---|---|---|---|---|---|---|---|---|---|---|---|---|---|---|---|
| | | | | | | | | | (mass %) | (mass %) | | | | | |
| | | | | | | | | | of parti- | of parti- | | | | | |
| | | | | | | | | | cles with | cles with | | | 1 mass % | | Coeffi- |
| | | | | | | | | | a particle | a particle | | | aqueous | | cient of |
| Ex./ | Proportion (mass %) of particles | | | | | | | | size of | size of | | Degree of | solution | Fria- | thermal |
| Comp. | remaining on each sieve (μm) | | | | | | | | 150 μm | 300 μm | Degree of | compres- | viscosity | bility | expansion |
| Ex. | 500 | 300 | 250 | 180 | 150 | 106 | 75 | Saucer | or more | or more | dispersion | sion | (mPa · s). | (%) | (%) |
| Ex. 1 | 0.0 | 4.7 | 1.7 | 11.7 | 24.2 | 37.8 | 14.2 | 5.8 | 42.3 | 4.7 | 12.3 | 8.9 | 11900 | 0.3 | 1.7 |
| Ex. 2 | 0.0 | 2.6 | 1.0 | 8.7 | 10.1 | 33.8 | 30.1 | 13.7 | 22.4 | 2.6 | 14.1 | 11.0 | 8000 | 0.1 | 1.3 |
| Comp. | 0.0 | 7.9 | 4.1 | 18.4 | 20.1 | 34.1 | 12.3 | 3.1 | 50.6 | 7.9 | 12.7 | 10.9 | 10980 | 1.3 | 1.8 |
| Ex. 1 | | | | | | | | | | | | | | | |
| Comp. | 0.0 | 13.6 | 11.6 | 32.9 | 14.4 | 15.0 | 7.5 | 5.0 | 72.5 | 13.6 | 15.1 | 7.5 | 15560 | 15.0 | 2.4 |
| Ex. 2 | | | | | | | | | | | | | | | |
| Comp. | 0.6 | 16.9 | 8.7 | 26.0 | 12.6 | 24.6 | 8.5 | 2.0 | 64.8 | 17.5 | 11.2 | 27.0 | 16060 | 6.5 | 5.9 |
| Ex. 3 | | | | | | | | | | | | | | | |
| Comp. | 4.4 | 5.6 | 2.7 | 10.1 | 8.4 | 15.7 | 45.7 | 7.4 | 31.2 | 10.0 | 15.3 | 12.5 | 16040 | 3.5 | 2.7 |
| Ex. 4 | | | | | | | | | | | | | | | |

Table 1 shows the results of measuring the particle size distribution of the polyalkylene oxide particles for a preparation produced in each of the Examples and Comparative Examples, and the results of measuring the 1 mass % aqueous solution viscosity, friability, degree of compression, degree of dispersion, and coefficient of thermal expansion.

The results of Examples 1 and 2 in Table 1 show that the tablets obtained from polyalkylene oxide particles in which the content of particles having a particle size of 150 μm or more is less than 46 mass %, and the content of particles having a particle size of 300 μm or more is less than 10 mass % had low friability. Further, by using the polyalkylene oxide particles, compression-molded articles with a low coefficient of thermal expansion were formed. Thus, it was found that the polyalkylene oxide particles obtained in the Examples are suitable for use in forming preparations that are less susceptible to chipping and cracking.

1. Polyalkylene oxide particles for a preparation,
wherein
the content of particles having a particle size of 150 μm or more is less than 46 mass %, and
the content of particles having a particle size of 300 μm or more is less than 10 mass %.

2. The polyalkylene oxide particles for a preparation according to claim 1, which have a 1 mass % aqueous solution viscosity of 40 to 20000 mPa·s.

3. The polyalkylene oxide particles for a preparation according to claim 1, which have a 1 mass % aqueous solution viscosity of less than 40 mPa·s and a 5 mass % aqueous solution viscosity of 30 to 50000 mPa·s.

4. A pharmaceutical composition comprising the polyalkylene oxide particles for a preparation according to claim 1.

5. A preparation composition comprising the pharmaceutical composition according to claim 4.

6. The preparation composition according to claim 5, which comprises the polyalkylene oxide particles in an amount of 20 mass % or more.

7. A preparation comprising the preparation composition according to claim 5.

8. The preparation according to claim 7, which comprises a compression-molded article of the preparation composition.
